(19) United States
(12) Patent Application Publication
(10) Pub. No.: US 2011/0126842 A1
(43) Pub. Date: Jun. 2, 2011

(54) Compacted Sexual-Transmitted-Disease Controller Brief

(76) Inventor: Michael Vasquez, San Pedro, CA (US)

(21) Appl. No.: 12/957,257

(22) Filed: Nov. 30, 2010

Related U.S. Application Data
(60) Provisional application No. 61/266,111, filed on Dec. 2, 2009.

(51) Int. Cl. A61F 6/04; A41B 9/02

(52) U.S. Cl. 128/844; 2/404

(57) ABSTRACT

A compacted sexual-transmitted-disease controller brief which encases the lower abdomen, pelvic region (including genitalia and anus) and upper thigh regions of a user. The compacted controller brief is comprised of a waist enclosure, a pelvic enclosure, left and right upper thigh enclosures and a condom enclosure that are each compacted and a folding arrangement that compacts each of the compacted enclosures. The compacted brief is suitable for a user to slide onto the user's pelvis region and to unravel and unfurl so as to extend about the users waist region, upper thighs and erect penis.

### CROSS-REFERENCE TO RELATED APPLICATIONS

This application claims priority from U.S. Provisional Application Ser. No. 61/266,111, filed on Dec. 2, 2009. The entirety of that provisional application is incorporated herein by reference.

### BACKGROUND OF THE INVENTION

1. Field of the Invention

This invention pertains generally to prophylactic-contraceptive devices and more particularly to briefs and shields that limit the transmission of disease producing microorganisms and fertilizing spermatozoa.

2. Related Art

It is known in the art to construct briefs and shields that limit the transmission of disease producing microorganisms and fertilizing spermatozoa during sexual interaction. Such devices are taught in U.S. Pat. Nos. 5,269,239, 4,993,433 and D419,233, Canadian Patent No. 201156981 and French Patent No. 2609888 which are incorporated by reference herein for the limited purpose of enablement and not for purposes of claim construction.

These prior art devices have disadvantages which may include being difficult to put on, being inconvenient to use, allowing the transfer of fluid, employing straps or bands, employing adhesive, being loosely draped and being expensive.

Deficiencies in the art are that devices have large packaging sizes, insufficiently protect against contracting a disease and/or pregnancy, are not disposable and/or are not recyclable.

There exists a need for a controller brief that is compacted into a small packaging and carrying size.

There is need for a controller brief that is easy to put on and convenient to use. There is a further need for a controller brief that does not employ straps, bands or adhesive.

There is need for a controller brief that provides enhanced shielding ability against contracting a disease and/or pregnancy. There is a further need for a controller brief that provides a greater protection area and/or is conformed fitting.

There is need for a controller brief that is readily disposable, disposable in an eco-friendly and/or recyclable.

There is need for a controller brief that is inexpensive.

The present invention satisfies these needs, as well as others, and generally overcomes the presently known deficiencies in the art.

### SUMMARY OF THE INVENTION

The present invention is directed to a compacted brief that controls sexual-transmitted-disease and limits pregnancy.

An object of the present invention is a controller brief that is compacted into a small packaging size.

Another object of the present invention is a controller brief that is easy to put on and convenient to use.

Another object of the present invention is a controller brief that does not employ straps, bands or adhesive.

Another object of the present invention is a controller brief that provides enhanced shielding ability against contracting a disease and/or pregnancy, provides a greater protection area and is conformed fitting.

Another object of the present invention is a controller brief that is readily disposable, disposable in an eco-friendly and/or recyclable.

Another object of the present invention is a controller brief that is inexpensive.

One aspect of the present invention is a compacted sexual-transmitted-disease controller brief. The controller brief has a waist enclosure that is compacted. The waist enclosure is an enclosure that is conformed for receiving the waist of a user. The waist enclosure is compacted by configuring it into one of a group of configurations. The compacted configuration can be a waist enclosure rolled in a ring, a waist enclosure rolled in a coil or a waist enclosure that has at least one longitudinal fold upon which it is bent over upon itself into an overlying relationship.

Inferior to and integral with the waist enclosure is a pelvic enclosure. The pelvic enclosure is conformed for receiving a user's pelvic region. The pelvic enclosure has an anterior region, posterior region and crotch.

Integral with and inferior to the pelvic enclosure, there is a left upper thigh enclosure that is compacted. The left upper thigh enclosure is anatomically positioned and conformed for receiving the left upper thigh of a user. The left upper thigh enclosure is compacted by configuring it into one of a group of configurations. The compacted configurations can be an upper thigh enclosure rolled in a ring, an upper thigh enclosure rolled in a coil and an upper thigh enclosure that has at least one longitudinal fold upon which it is bent over upon itself into an overlying relationship.

Integral with and inferior to the pelvic enclosure, there is a right upper thigh enclosure that is compacted. The upper right thigh enclosure is anatomically positioned and conformed for receiving the right upper thigh of a user. The right upper thigh enclosure is compacted by configuring it into one of a group of configurations. The compacted configurations can be an upper thigh enclosure rolled in a ring, an upper thigh enclosure rolled in a coil and an upper thigh enclosure that has at least one longitudinal fold upon which it is bent over upon itself into an overlying relationship.

There is a condom enclosure that is compacted. The condom enclosure is integral with the anterior region of the pelvic enclosure and anatomically positioned and conformed for receiving the erect penis of a user. The condom enclosure is in a compacted configuration. The compacted can be a condom enclosure rolled in a ring, a condom enclosure rolled in a coil and a condom enclosure that has at least one cross-sectional fold upon which it is bent over upon itself into an overlying relationship.

There is a folding arrangement upon which are brought into compact storage configuration the waist enclosure that is compacted, the pelvic enclosure, the upper thigh enclosures that are compacted and the condom enclosure that is compacted.

The compacted brief is suitable for a user to slide the compacted brief onto the user's pelvis region, unravel the waist enclosure so as to extend along the users waist region, unravel the upper thigh enclosures so as to extend along the user's upper thighs and where the condom enclosure unfurls about a user's erect penis.

In another aspect of the invention the aforementioned folding arrangement is comprised of comprised of one or more cross-sectional folds approximately parallel to an axis the runs from the crotch to center of the waist enclosure and one or more longitudinal folds that are approximately perpendicular to the cross-sectional folds.

In another of the invention the aforementioned folding arrangement is comprised of a first fold that is a cross-sectional and medial fold that doubles over the pelvic enclosure and compacted waist enclosure upon themselves and stacks the compacted right upper thigh enclosure over the compacted left upper thigh enclosure; a second fold that is a longitudinal and medial fold which folds over upon itself the doubled over pelvic enclosure, stacks the crotch region over the doubled over compacted waist enclosure and doubles over upon themselves the stacked right and left compacted upper thigh enclosures and a third fold that is a cross-sectional and medial fold relative to the foregoing partially folded controller brief and brings the controller brief into compact storage configuration.

The previously described versions of the present invention has many advantages which include a small packaging size, applied by stepping into the controller brief and rolling on portions, being easy to put on it and an enhanced barrier against disease transmission and pregnancy.

### BRIEF DESCRIPTION OF THE DRAWINGS

These and other features, aspects and advantages of the present invention will become better understood with reference to the following description, appended claims and accompanying drawings where:

FIG. 1 shows a perspective view of an expanded controller brief according to the present invention;

FIG. 2A shows a front planar view of a controller brief having compacted waist and leg enclosures and a non-compacted condom enclosure, along with a mid-sectional fold line, all according to the present invention;

FIG. 2B shows a front planar view of a partially compacted controller brief having compacted waist and leg enclosures with a partially compacted condom enclosure having been folded at its midline, along with a cross-sectional fold line, all according to the present invention;

FIG. 2C shows a front planar view of a partially controller brief having compacted waist and leg enclosures with a partially compacted condom enclosure having been folded at its midline and cross-sectional line, along with a cross-sectional fold line, all according to the present invention;

FIG. 2D shows a front planar view of a partially controller brief having compacted waist and leg enclosures with a partially compacted condom enclosure having been folded three times along midline and cross-sectional lines, all according to the present invention;

FIG. 2E shows a front planar view of a partially compacted controller brief having compacted waist and leg enclosures with a partially compacted condom enclosure having been folded three times along midline and cross-sectional lines, along with two fold lines, all according to the present invention;

FIG. 2F shows a front planar view of a partially compacted controller brief having compacted waist and leg enclosures with a non-compacted condom enclosure having been folded three times along midline and cross-sectional lines, along with two fold lines, all according to the present invention;

FIG. 2E shows a front planar view of a compacted controller brief, according to the present invention;

FIG. 3A shows a cross-sectional of an enclosure that is folded according to the present invention;

FIG. 3B shows a cross-sectional of an enclosure that is coiled in a ring according to the present invention;

FIG. 3C shows a cross-sectional of an enclosure that is rolled according to the present invention;

FIG. 4 shows a frontal perspective of a controller brief having compacted waist and leg enclosures and a non-compacted condom enclosure, along with a plurality of fold lines, all according to the present invention;

FIG. 5 shows a frontal perspective of a controller brief having compacted waist, leg and condom enclosures, according to the present invention;

FIG. 6 shows a frontal perspective of a controller brief having compacted waist, leg and unfurled condom enclosure being worn by a user, along with arrows to indicate expanding of compacted enclosures, all according to the present invention and

FIG. 7 shows a perspective view of an expanded controller brief being worn by a user according to the present invention.

### DETAILED DESCRIPTION OF THE INVENTION

The present invention is described more fully in the following disclosure. In this disclosure, there is a discussion of embodiments of the invention and references to the accompanying drawings in which embodiments of the invention are shown. These specific embodiments are provided so that this invention will be understood by those skilled in the art. This invention is not limited to the specific embodiments set forth herein below and in the drawings. The invention is embodied in many different forms and should be construed as such with reference to the appended claims.

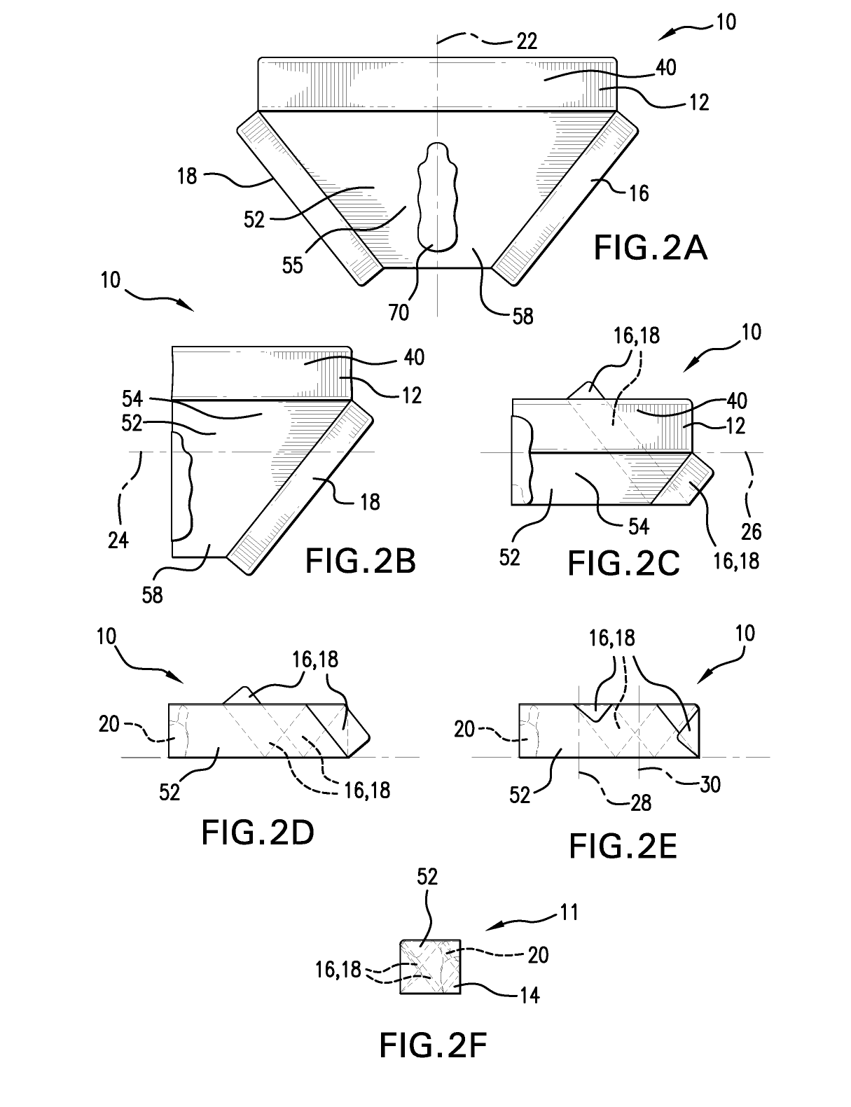

Referring to FIG. 7, when worn by a user, the controller brief (10) encases the lower abdomen, pelvic region including genitalia and anus, and upper thigh regions of a user. Referring to FIGS. 2 and 5, the compacted controller brief (10) is comprised of a waist enclosure that is compacted (12), pelvic enclosure that is compacted (14), left upper thigh enclosure that is compacted (16), right upper thigh enclosure that is compacted (18), a condom enclosure that is compacted (20) and a folding arrangement (22, 24, 26, 28, 30, 32 and 33)

The controller brief (10) is formed from a flexible, elastic and impermeable material. The material substantially restricts the passage of spermatozoa and microbes. Preferably, the material is also soft. Suitable materials are rubber, polyvinyl chloride, latex, silicone and polyurethane. A preferred material is latex. A preferred material is thermoplastic or other material that can be recycled so at to be environmentally friendly. Typically, the thickness of the material is between 0.5 mils to 100 mils and preferably 1 to 5 mils.

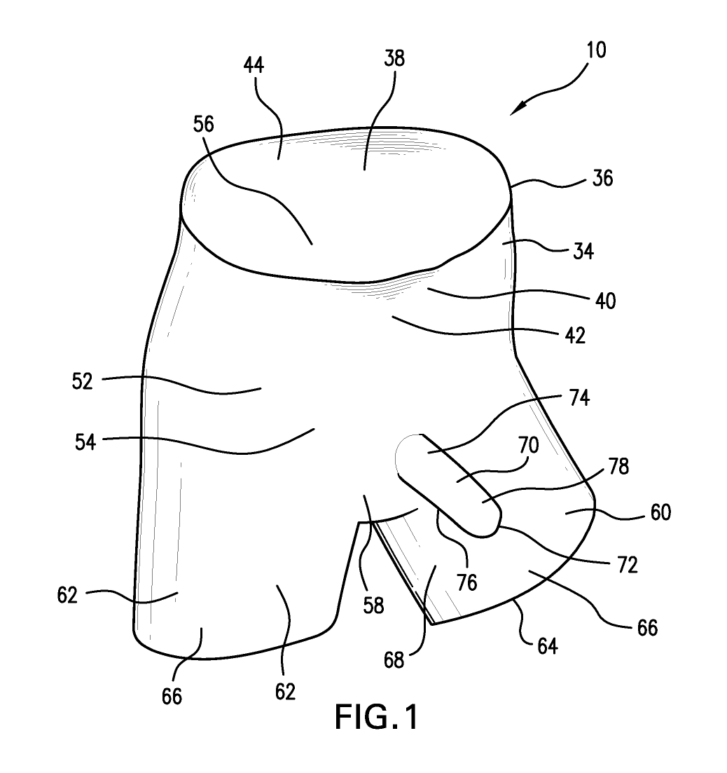

Referring to FIG. 1, the waist enclosure (34) is a circular band or cylinder and is conformed for receiving a waist of an user. The cross-section generally has a substantially annular or ring shape. The waist enclosure (34) has a top edge (36), a posterior region (38), an anterior region (40), an external surface (42) and an internal surface (44).

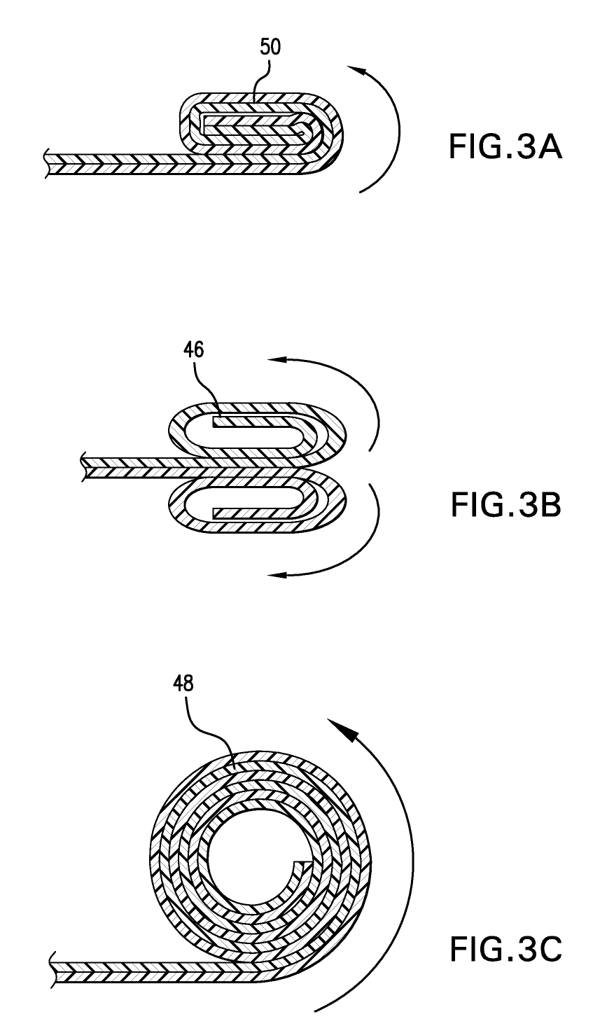

Referring to FIGS. 3A, 3B and 3C, the waist enclosure that is compacted (12) typically has one of three configurations; in particular, coiled in a ring (46), rolled in a coil (48) and a waist enclosure that has at least one longitudinal fold upon which it is bent over upon itself into an overlying relationship (50). Referring to FIG. 3B, the waist enclosure coiled in a ring (46) can be formed by beginning at the top edge (36) and doing a 360 degree roll up of the waist enclosure (34) into a ring either over rolling over the external surface (42) or by rolling over the internal surface (44). Referring to FIG. 3C, the waist enclosure rolled in a coil (48) can be formed by beginning at the top edge (36) and rolling up the waist enclosure (34) into a coil either rolling over the external surface (42) of the posterior region (38) or by rolling over the external surface (42) of the anterior region (40).

Referring to FIG. 3A, the waist enclosure (34) which it is bent over upon itself into an overlying relationship (50) can be formed by lying the waist enclosure (34) flat with the anterior and posterior regions (34,38) of the waist enclosure (34) overlying each other in a bilayer. Typically there is between about 1 to about 5 longitudinal fold lines that are substantially parallel to the top edge (36) and equidistant apart. In the alternative, the waist enclosure can be successively folded in about half along fold lines that are substantially parallel to the top edge (36). Preferably there are about 3 to about 5 longitudinal folds and most preferably there is about 3 longitudinal folds (32). In doing the bending, the waist enclosure (34) is typically bent over upon itself by folding the bilayer, or multiple bilayers that have brought in overlying relation from a prior fold, about the external surface (42) of either the anterior region (40) or the posterior region (38) of the waist enclosure (34).

Referring to FIG. 1, the pelvic enclosure (52) is an enclosure conformed to receive the pelvic region of a user. Typically, the pelvic enclosure (52) has the general shape of a bikini style brief or a boxer style brief with the general shape of a bikini style brief is preferred. The pelvic enclosure (52) is integral with and inferior to the waist enclosure (34). The pelvic enclosure has an anterior region (54) that orients over the genital region of a user and a posterior region (56) that orients over the buttock region of the user and a crotch region (58).

Referring to FIG. 1, the left upper thigh enclosure (60) and the right upper thigh enclosure (62) are typically circular bands or cylinders and conformed for receiving the upper thigh of a user. The cross-section generally has a substantially annular or ring shape. The left upper thigh enclosure (60) and the right upper thigh enclosure (62) are each integral with and inferior to the pelvic enclosure (52) and anatomically positioned for receiving a thigh of a user.

Referring to FIGS. 3A, 3B and 3C, the upper thigh enclosures (60, 62) that is compacted typically has one of three configurations; in particular, a coiled in a ring (46), rolled in a coil (48) and a fold arrangement comprised of at least one longitudinal fold upon which it is bent over upon itself into an overlying relationship (50). Referring to FIG. 3B, the thigh enclosure coiled in a ring (46) can be formed by beginning at the outer edge (64) and doing a 360 degree roll up of the thigh enclosures (60, 62) into a ring either by rolling over the external surface (66) or by rolling over the internal surface of the thigh enclosure (60,62). Referring to FIG. 3C, the thigh enclosure rolled in a coil (48) can be formed by beginning at the outer edge (64) and rolling up the thigh enclosure into a coil either by rolling over the external surface (66) of the anterior region (68).

Referring to FIG. 3A, the thigh enclosures (60, 62) which it is bent over upon itself into an overlying relationship (50) can be formed by lying the thigh enclosure flat with the anterior region (68) and posterior regions of the thigh enclosure (60, 62) overlying each other in a bilayer. Typically there is between about 1 to about 5 longitudinal fold lines that are substantially parallel to the outer edge (64) and about equidistant apart. In the alternative, the thigh enclosures (60, 62) can be successively folded in about half along fold lines that are substantially parallel to the outer edge (64). Preferably there are about 3 to about 5 longitudinal folds and most preferably there is about 3 longitudinal folds. In doing the bending, the thigh enclosure (60, 62) is typically bent over upon itself by folding the bilayer, or multiple bilayers that have brought in overlying relation from a prior fold, about the external surface (66) of either the anterior region (68) or the posterior region of the thigh enclosures (60, 62).

Referring to FIG. 1, the condom enclosure (70) typically has the shape of a closed ended tube or closed ended cylinder and conformed for receiving a penis of a user. The condom enclosure (70) is integral with the anterior region (54) of the pelvic enclosure (52) and anatomically positioned on the anterior region (54) of the pelvic enclosure (52) for receiving the erect penis of a user. The condom enclosure has a closed end (72), a superior region (74), an inferior region (76), an external surface (78), an internal surface (44) and an axis. Optionally, it can have a ribbed outer surface or other texture, have a reservoir tip, have a lubricant coating and/or a spermicidal coating. The material thickness can thinned to increase sensitivity at the expense of increasing the risk of breakage or rupture.

Referring to FIGS. 3A and 3C, the condom enclosure that is compacted (20) typically has one of two configurations; in particular, a condom enclosure rolled in a coil (48) and a condom enclosure that has at least one longitudinal fold upon which it is bent over upon itself into an overlying relationship (50). Referring to FIG. 3C, the condom enclosure (70) can be rolled into a ring (48) by first doubling the condom enclosure (70) over upon itself by traveling the closed end (72) down the internal surface until the juncture with the pelvic enclosure (52) so as to create an open-ended by bilayer tube or cylinder. The condom enclosure (70) is rolled in a coil (48) beginning at the closed end (72) and rolling up the condom enclosure (70) into a coil. The rolling can done at any orientation. It is preferred that the rolling be along the superior region (74) or the inferior region (76) for easy unfurling.

Referring to FIG. 3A, the condom enclosure (70) which it is bent over upon itself into an overlying relationship (50) typically has between about 1 to about 10 cross-sectional folds. The cross-sectional folds can be equidistant apart and can form a serpentine/zig-zag folding pattern. In the alternative, the condom enclosure can be successively folded in about half along cross-sectional fold lines. Preferably there is a sufficient number of folds to reduce the condom enclosure so that it rests substantially flat against the pelvic enclosure in a small area.

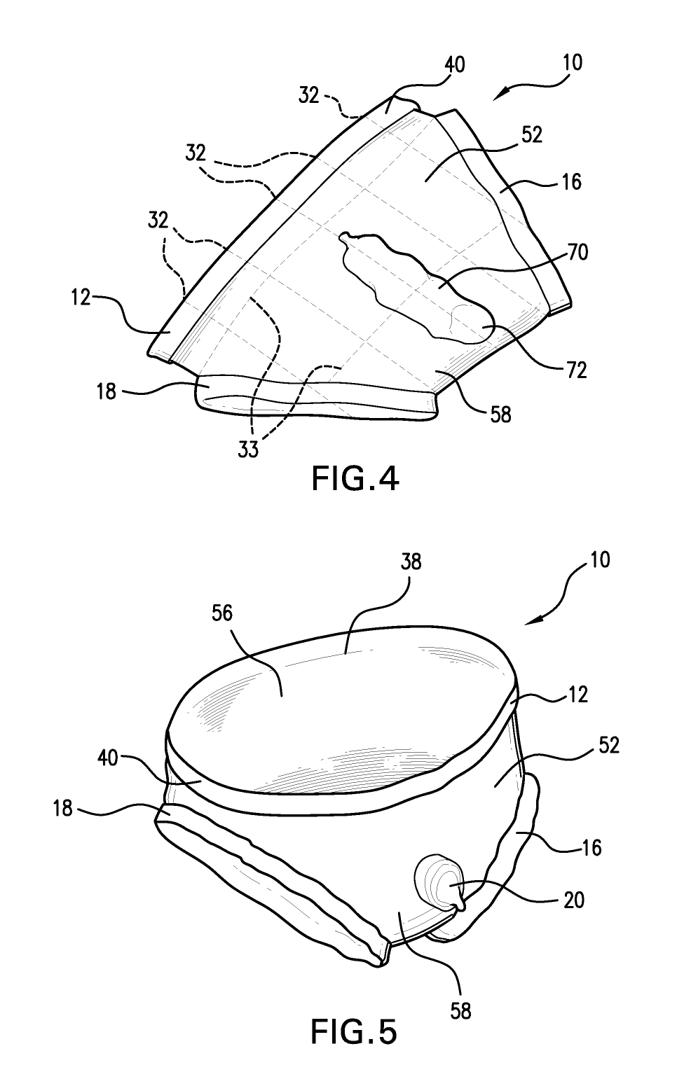

Referring to FIGS. 2 and 4, in a preferred embodiment where the condom enclosure (70) bent over upon itself into an overlying relationship (50), this folding is in conjunction with the folding of the controller brief as a whole into a compact configuration (11), as discussed below. Accordingly, some or all of the folds of the condom enclosure (70) are the folds (22, 24, 26, 28, 30 and 32) made in folding the controller brief as a whole into a compact configuration (11).

Referring to FIGS. 2 and 4, there is a folding arrangement (22, 24, 26, 28, 30 and 32) by which are brought into compact storage configuration the compacted waist enclosure (12), the pelvic enclosure (52), the compacted upper thigh enclosures (16,18) and the compacted condom enclosure (20) or un-compacted condom enclosure (70). This folding arrangement (22, 24, 26, 28, 30 and 32) can be comprised of one or more longitudinal folds (22, 28, 30 and 32) approximately parallel to an axis the runs from the midpoint of the crotch (58) to mid-point of the waist enclosure (34) when the controller brief (10) is laid flat one or more cross-sectional folds (24, 26 and 33) that are approximately perpendicular to the foregoing.

Continuing to refer to FIG. 2, in a preferred folding arrangement, the controller brief (10) lies flat with the anterior and posterior regions of the pelvic enclosure (54, 56) overlying each other in a bilayer. Referring to FIG. 2A, there is a first fold (22) that is a longitudinal and medial fold (22) that doubles over the pelvic enclosure (52) and compacted waist enclosure (12) upon themselves and stacks the compacted right upper thigh enclosure (18) over the compacted left upper thigh enclosure (16). Referring to FIG. 2B, there is a second fold that is a cross-sectional fold (24) which folds over upon itself the doubled over pelvic enclosure (52), stacks the crotch region (42) over the doubled over compacted waist enclosure (12) and doubles over upon themselves the stacked right and left compacted upper thigh enclosures (16, 18). Referring to FIGS. 2C and 2D, there is a third fold (26) that is a cross-sectional and medial fold relative to the foregoing partially folded controller brief (10). Referring to FIGS. 2E and 2F, there are successive folds (28, 30) and brings the controller brief into a compact storage configuration (11).

Referring to FIG. 4, in alternative embodiments, there are additional cross-sectional and longitudinal folds (32, 33) beyond the foregoing folds (22, 24, 26, 28, and 30) to compact the controller brief (11) to a desired size.

The controller brief (10) is manufactured and formed by molding technology known in the art for making standard condoms and shields.

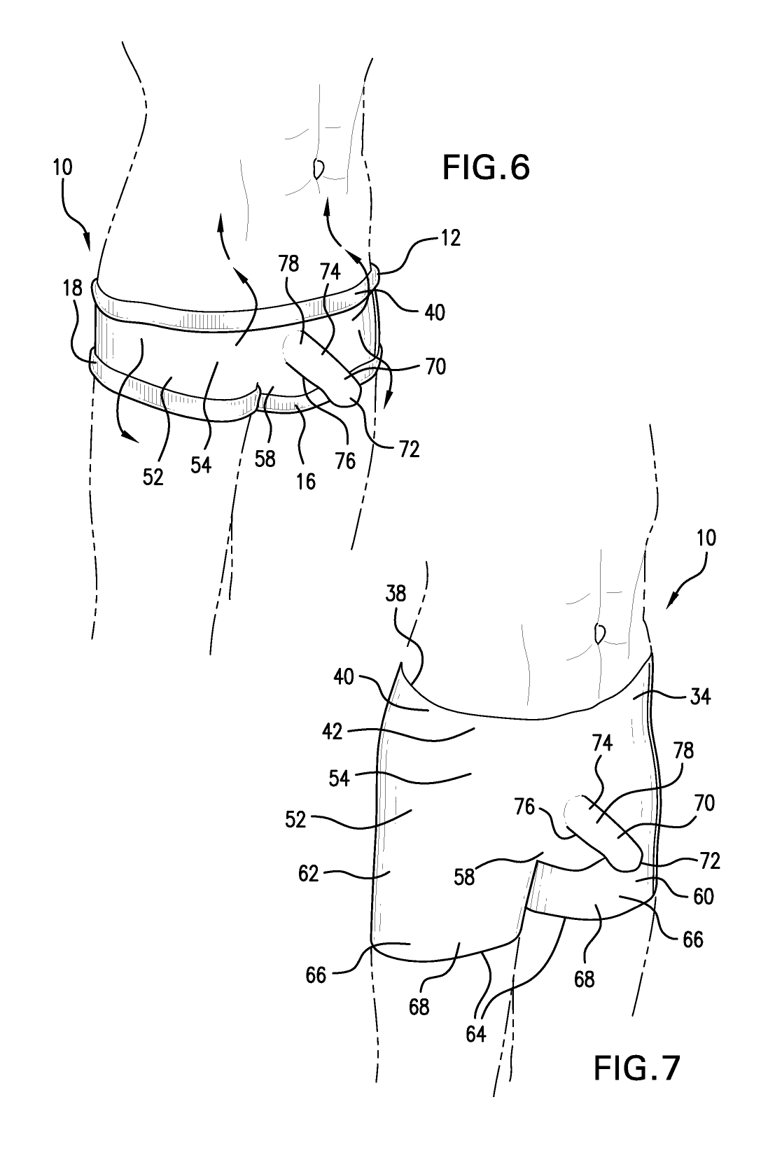

Referring to FIGS. 6 and 7, a user puts on the compacted controller brief (11) by stepping into it and sliding it up the users legs onto the user's pelvic region. The user in an order that pleases the user unravels the waist enclosure (12) so as to extend it (34) along the user's waist region, unravels the upper thigh enclosures (16, 18) so as to extend them along the user's upper thighs (60, 62) and the condom enclosure (20) can unfurl (70) about a user's erect penis or be manipulated to achieve the same. When worn, the controller brief (10) remains in substantially stable position on a user do to the contraction of force of the expanded elastic material from which the controller brief (10) is formed.

In an alternative embodiment, the controller brief (10) is suitable for disposal by being flushed down a toilet. To be efficaciously flushable, the controller brief is made from a material having a thickness that is sufficiently thin that the overall size and mass of the controller brief can flush down and pass through drain pipes without clogging.

In an alternative embodiment, the controller brief (10) is suitable for cleaning and reuse.

In an alternative embodiment, the controller brief (10) is decorated with an illustration; for example, a baseball scene or a NASCAR racing car scene. The illustration can be printed on the brief.

In an alternative embodiment, the controller brief (10) is an attractive color. The controller brief (10) can be colored by adding a dye into the material from which the controller brief is formed or by applying a colorant to its surface.

In an alternative embodiment, the controller brief (10) is of varying sizes so to accommodate different size users.

In preferred embodiment the compacted controller brief (10) is packaged into an envelope or sleeve.

The previously described versions of the present invention have many advantages. One advantage is that the controller brief completely encases the lower abdominal, pelvic and upper thigh regions of users so as to limit skin to skin contact and significantly restrict the transmission of bodily fluid and disease. Those inflicted with sexually transmittable diseases often times have open sores, wounds, rashes, pimples and other skin disorders in the lower abdominal, pelvic and thigh regions through which disease is transmitted.

Another advantage of the controller brief encasing the lower abdominal, pelvic and upper thigh regions and being formed from an elastic material is that the controller brief does not slip off or move into an uncomfortable position during sexual activity.

Another advantage is a small packaging size. A related advantage is that the controller brief can be easily carried and transported by itself or in say a wallet.

### EXAMPLES

The following examples further describe and demonstrate embodiments within the scope of the present invention. The examples are given solely for the purpose of illustration and are not to be construed as limitations or restrictions of the present invention, as persons skilled in the art will quickly realize many variations thereof are possible that are all within the spirit and scope of the invention.

### Example 1

In a hypothetical example, the controller brief is made from a material having a thickness that is between about 0.5 mils to 100 mils.

### Example 2

In a hypothetical example, the controller brief is made from a material having a thickness that is between about 1 mil to 5 mils.

Although the present invention has been described in considerable detail with reference to certain preferred versions thereof, other versions are possible with substituted, varied and/or modified materials and steps are employed. These other versions do not depart from the invention. Therefore, the spirit and scope of the appended claims should not be limited to the description of the preferred versions contained herein.

What is claimed is:

1. A compacted sexual-transmitted-disease controller brief comprised of:
a. a waist enclosure conformed for receiving the waist of a user and that is compacted in a configuration selected from the group consisting of rolled in a ring, rolled in a coil and folded longitudinally over upon itself into an overlying relationship;
b. a pelvic enclosure conformed for receiving the pelvic region of a user that is integral with and inferior to the waist enclosure and has an anterior region, posterior region and crotch;
c. a left upper thigh enclosure that is integral with and inferior to the pelvic enclosure and anatomically positioned and conformed for receiving the left upper thigh of a user that is compacted in a configuration selected from the group consisting of rolled in a ring, rolled in a coil and folded longitudinally over upon itself into an overlying relationship;
d. a right upper thigh enclosure that is integral with and inferior to the pelvic enclosure and anatomically positioned and conformed for receiving the right upper thigh of a user that is compacted in a configuration selected from the group consisting of rolled in a ring, rolled in a coil and folded longitudinally over upon itself into an overlying relationship;
e. a condom enclosure that is integral with the anterior region of the pelvic enclosure and anatomically positioned and conformed for receiving the erect penis of a user that is compacted in a configuration selected from the group consisting of rolled in a ring, rolled in a coil and folded longitudinally over upon itself into an overlying relationship and
f. a folding arrangement upon which are brought into compact storage configuration the waist enclosure that is compacted, the pelvic enclosure, the upper thigh enclosures that are compacted and the condom enclosure that is compacted,
where the compacted brief is suitable for a user to slide the compacted brief onto the user's pelvis region, unravel the waist enclosure so as to extend along the users waist region, unravel the upper thigh enclosures so as to extend along the user's upper thighs and where the condom enclosure unfurls about a user's erect penis.

2. The controller brief of claim 1 made out of a flexible and impermeable material that substantially restricts the passage of spermatozoa and microbes.

3. The controller brief of claim 1 where the folding arrangement is comprised of one or more longitudinal folds approximately parallel to an axis the runs from the crotch to center of the waist enclosure and one or more cross-sectional folds that are approximately perpendicular to the longitudinal folds.

4. The controller brief of claim 1 where the anterior and posterior regions of the pelvic enclosure overly each other in a bilayer and the folding arrangement is comprised of:
a. a first fold that is a longitudinal and medial fold that doubles over the pelvic enclosure and compacted waist enclosure upon themselves and stacks the compacted right upper thigh enclosure over the compacted left upper thigh enclosure;
b. a second fold that is a cross-sectional and medial fold which folds over upon itself the doubled over pelvic enclosure, stacks the crotch region over the doubled over compacted waist enclosure and doubles over upon themselves the stacked right and left compacted upper thigh enclosures and
c. a third fold that is a cross-sectional and medial fold relative to the foregoing partially folded controller brief and brings the controller brief into compact storage configuration.

5. A compacted sexual-transmitted-disease controller brief comprised of:
a. a waist enclosure that is:
i. flexible and substantially impermeable to the passage of spermatozoa and microbes;
ii. conformed for receiving the waist of a user and
iii. compacted in a configuration selected from the group consisting of rolled in a ring, rolled in a coil and folded longitudinally over upon itself into an overlying relationship;
b. a pelvic enclosure that is:
i. flexible and substantially impermeable to the passage of spermatozoa and microbes;
ii. conformed for receiving the pelvic region of a user;
iii. integral with and inferior to the waist enclosure and
iv. has an anterior region, posterior region and crotch;
c. a left upper thigh enclosure that is:
i. flexible and substantially impermeable to the passage of spermatozoa and microbes;
ii. integral with and inferior to the pelvic enclosure;
iii. anatomically positioned and conformed for receiving the left upper thigh of a user and
iv. is compacted in a configuration selected from the group consisting of rolled in a ring, rolled in a coil and folded longitudinally over upon itself into an overlying relationship;
d. a right upper thigh enclosure that is:
i. integral with and inferior to the pelvic enclosure;
ii. anatomically positioned and conformed for receiving the right upper thigh of a user and
iii. compacted in a configuration selected from the group consisting of rolled in a ring, rolled in a coil and folded longitudinally over upon itself into an overlying relationship;
e. a condom enclosure that is:
i. flexible and substantially impermeable to the passage of spermatozoa and microbes;
ii. integral with the anterior region of the pelvic enclosure;
iii. anatomically positioned and conformed for receiving the erect penis of a user and
iv. compacted in a configuration selected from the group consisting of rolled in a ring, rolled in a coil and folded longitudinally over upon itself into an overlying relationship and
f. a folding arrangement comprised of:
i. one or more longitudinal folds approximately parallel to an axis the runs from the crotch to center of the waist enclosure and
ii. one or more cross-sectional folds that are approximately perpendicular to the longitudinal folds,
upon which are brought into compact storage configuration the waist enclosure that is compacted, the pelvic enclosure, the upper thigh enclosures that are compacted and the condom enclosure that is compacted,
where the compacted brief is suitable for a user to slide the compacted brief onto the user's pelvis region, unravel the waist enclosure so as to extend along the users waist region, unravel the upper thigh enclosures so as to extend along the user's upper thighs and where the condom enclosure unfurls about a user's erect penis.

6. The controller brief of claim 5 where:
a. the waist enclosure is compacted in a rolled in a ring configuration;
b. the upper thigh enclosures are compacted in a rolled in a ring configuration and
c. the condom enclosure is compacted in a rolled a ring configuration.

7. The controller brief of claim 5 where:
a. the waist enclosure is compacted in a rolled in a coil configuration;
b. the upper thigh enclosures are compacted in a rolled in a coil configuration and
c. the condom enclosure is compacted in a rolled a coil configuration.

8. The controller brief of claim 5 where:
a. the waist enclosure is compacted in a folded longitudinally over upon itself configuration;
b. the upper thigh enclosures are compacted in a folded longitudinally over upon itself configuration and
c. the condom enclosure is compacted in a folded longitudinally over upon itself configuration.

9. The controller brief of claim 5 where:
a. the waist enclosure is compacted in a rolled in a ring configuration;
b. the upper thigh enclosures are compacted in a rolled in a ring configuration and
c. the condom enclosure is compacted in a folded longitudinally over upon itself configuration.

10. The controller brief of claim 5 where:
a. the waist enclosure is compacted in a rolled in a coil configuration;
b. the upper thigh enclosures are compacted in a rolled in a coil configuration and
c. the condom enclosure is compacted in a folded longitudinally over upon itself configuration.

11. A compacted sexual-transmitted-disease controller brief comprised of:
a. a waist enclosure that is:
i. flexible and substantially impermeable to the passage of spermatozoa and microbes;
ii. conformed for receiving the waist of a user and
iii. compacted in a configuration selected from the group consisting of rolled in a ring, rolled in a coil and folded longitudinally over upon itself into an overlying relationship;
b. a pelvic enclosure that is:
i. flexible and substantially impermeable to the passage of spermatozoa and microbes;
ii. conformed for receiving the pelvic region of a user;
iii. integral with and inferior to the waist enclosure;
iv. has an anterior region, posterior region and crotch and
v. the anterior and posterior regions overly each other in a bilayer
c. a left upper thigh enclosure that is:
i. flexible and substantially impermeable to the passage of spermatozoa and microbes;
ii. integral with and inferior to the pelvic enclosure;
iii. anatomically positioned and conformed for receiving the left upper thigh of a user and
iv. is compacted in a configuration selected from the group consisting of rolled in a ring, rolled in a coil and folded longitudinally over upon itself into an overlying relationship;
d. a right upper thigh enclosure that is:
i. integral with and inferior to the pelvic enclosure;
ii. anatomically positioned and conformed for receiving the right upper thigh of a user and
iii. compacted in a configuration selected from the group consisting of rolled in a ring, rolled in a coil and folded longitudinally over upon itself into an overlying relationship;
e. a condom enclosure that is:
i. flexible and substantially impermeable to the passage of spermatozoa and microbes;
ii. integral with the anterior region of the pelvic enclosure;
iii. anatomically positioned and conformed for receiving the erect penis of a user and
iv. compacted in a configuration selected from the group consisting of rolled in a ring, rolled in a coil and folded longitudinally over upon itself into an overlying relationship and
f. a folding arrangement comprised of:
i. a first fold that is a longitudinal and medial fold that doubles over the pelvic enclosure and compacted waist enclosure upon themselves and stacks the compacted right upper thigh enclosure over the compacted left upper thigh enclosure;
ii. a second fold that is a cross-sectional and medial fold which folds over upon itself the doubled over pelvic enclosure, stacks the crotch region over the doubled over compacted waist enclosure and doubles over upon themselves the stacked right and left compacted upper thigh enclosures and
iii. a third fold that is a cross-sectional and medial fold relative to the foregoing partially folded controller brief, such that the controller brief is in a compact storage configuration,
where the compacted brief is suitable for a user to slide the compacted brief onto the user's pelvis region, unravel the waist enclosure so as to extend along the users waist region, unravel the upper thigh enclosures so as to extend along the user's upper thighs and where the condom enclosure unfurls about a user's erect penis.

12. The controller brief of claim 11 where:
a. the waist enclosure is compacted in a rolled in a ring configuration;
b. the upper thigh enclosures are compacted in a rolled in a ring configuration and
c. the condom enclosure is compacted in a rolled a ring configuration.

13. The controller brief of claim 11 where:
a. the waist enclosure is compacted in a rolled in a coil configuration;
b. the upper thigh enclosures are compacted in a rolled in a coil configuration and
c. the condom enclosure is compacted in a rolled a coil configuration.

14. The controller brief of claim 11 where:
a. the waist enclosure is compacted in a folded longitudinally over upon itself configuration;
b. the upper thigh enclosures are compacted in a folded longitudinally over upon itself configuration and
c. the condom enclosure is compacted in a folded longitudinally over upon itself configuration.

15. The controller brief of claim 11 where:
a. the waist enclosure is compacted in a rolled in a ring configuration;
b. the upper thigh enclosures are compacted in a rolled in a ring configuration and
c. the condom enclosure is compacted in a folded longitudinally over upon itself configuration.

16. The controller brief of claim 11 where:
a. the waist enclosure is compacted in a rolled in a coil configuration;
b. the upper thigh enclosures are compacted in a rolled in a coil configuration and
c. the condom enclosure is compacted in a folded longitudinally over upon itself configuration.
